(19) United States
(12) Patent Application Publication
(10) Pub. No.: US 2025/0213442 A1
(43) Pub. Date: Jul. 3, 2025

(54) DAILY USE PRESERVATIVE-FREE COSMETIC COMPOSITIONS

(71) Applicant: LightWater Biosciences, Inc., Burlington, MA (US)

(72) Inventor: Soo-Young Kang, Burlington, MA (US)

(21) Appl. No.: 18/849,609

(22) PCT Filed: Mar. 24, 2023
(86) PCT No.: PCT/US2023/016210

§ 371 Date: Sep. 23, 2024

Related U.S. Application Data
(60) Provisional application No. 63/323,273, filed on Mar. 24, 2022.

(51) Int. Cl. A61K 8/34; A61K 8/365; A61K 8/99; A61Q 19/00

(52) U.S. Cl. CPC A61K 8/345; A61K 8/365; A61K 8/99; A61Q 19/00; A61K 2800/30; A61K 2800/522

(57) ABSTRACT

Provided herein are preservative-free aqueous cosmetic compositions.

### RELATED APPLICATIONS

This application claims the benefit of priority to U.S. Provisional Application No. 63/323,273 filed Mar. 24, 2022, the entire contents of which are incorporated herein by reference.

### BACKGROUND

Cosmetic formulation compositions require water as one of the main ingredients to create various aqueous water-based emulsions except for anhydrous (e.g., bar soaps) and oil mixture-based products formulated with 0% water. Aqueous cosmetic formulations consist of one-phase solutions and more commonly used two-phase emulsions (oil-in-water, water-in-oil), three-phase emulsions (water-oil-water, oil-water-oil), and other complex emulsion structures involving water as an ingredient. Water is used in aqueous cosmetic formulations as a solvent (a) to dissolve water-soluble ingredients, (b) to improve formulation blending. (c) to enhance formulation flowability, (d) to enhance application spreadability, (c) to improve formulation aesthetics, and (f) to impart moisturization/hydration to the skin. Additionally, cosmetic ingredients or raw materials are often in the form of blend materials containing water as a solvent or carrier for other excipients within a blend. The concentration of water in aqueous cosmetic compositions is often the highest among all ingredients by weight, as listed as the first ingredient in the cosmetic product packs. The aqueous cosmetic formulation has a risk of microbial growth and contamination. It is a current industry practice that manufacturers put preservative chemicals into aqueous cosmetic formulations to prevent microbial growth during the supply chain, storage, transport, and consumer usc.

Cosmetic products on the market today contain preservatives to suppress microbial growth during 2-3 years of the supply chain and consumer use, resulting in three major problems; (a) preservatives are one of the main skin irritation and sensitizers, (b) commonly used cosmetic active ingredients degrade over time rendering them ineffective during the consumer use cycle. Many skin-beneficial actives such as retinol and vitamin C are known to be chemically unstable. These actives break down and lose efficacy over this long storage and use period. As a result, consumers do not truly benefit from these actives, (c) preservatives may disrupt the skin's microbiome balance, which is essential for healthy skin [Wang et al., Journal of Cosmetic Dermatology, 2019; 18:652-658]. A report says the average woman uses 16 cosmetic products every day [https://www.telegraph.co.uk/beauty/face/essential-skincare-makeup-products-use-everyday/]. Each product a person puts on their face has its preservatives. Hence, many consumers are layering preservatives multiple times on a daily basis. This repeated overuse of preservatives would disrupt the microbiome balance, which is the skin's natural defense.

Cosmetic formulations are manufactured in industry-scale bulk for a year's worth of inventory. After packaging, the finished products are stored in warehouses for further distribution to fulfillment centers or retailers such as Sephora, Ulta, Department stores, CVS, Walmart, Amazon, etc. Cosmetic products are stored for a certain period again on retailers' shelves and warehouses until they are sold to consumers. After purchase, consumers use the products for months to years. For instance, cosmetic products show a label of a 12-month use date after opening.

Cosmetic products are stored for a considerable amount of time and used by consumers for months to years. This long cycle puts cosmetic products at risk of microbial growth and contamination. These products need a long shelf life for the supply chain of transport, inventory, distribution, and consumer use period. Therefore, current cosmetic products are generally “aged” and “stale” at the point of consumer purchase. Furthermore, during the consumer use period, cosmetic products are exposed to contaminants through the exposure to light, UV, air, and touching by finger and are susceptible to microbial growth. Therefore, current market cosmetic products are not “fresh.”

### SUMMARY

Provided herein are preservative-free aqueous cosmetic compositions.

The disclosed compositions are fresh, non-stale, non-irritating, and/or skin-microbiome-friendly cosmetic products.

### BRIEF DESCRIPTION OF THE FIGURES

FIG. 1A shows dry patch, redness, swelling, and skin lesions on the skin of a subject prior to applying the disclosed compositions.

FIG. 1B shows the treatment results (smooth, even texture and tone, and no skin lesions) on the subject's skin in FIG. 1A after applying the disclosed compositions for a period of time.

FIG. 2A shows redness, irritated and inflamed lesions on a subject with extremely sensitive skin prior to applying the disclosed compositions.

FIG. 2B shows the treatment results (smooth, even texture and tone, and no skin lesions) on the subject's skin in FIG. 2A after applying the disclosed compositions for a period of time.

### DETAILED DESCRIPTION

In one aspect, provided are preservative-free aqueous cosmetic compositions comprising distilled water.

In one aspect, the preservative-free aqueous cosmetic compositions are in the form of emulsions of the oil/water, water/oil, water-oil-water, or oil-water-oil type.

In one aspect, the preservative-free aqueous cosmetic compositions comprise distilled water at a concentration greater than or equal to 20% relative to the total weight of the composition.

In one aspect, the preservative-free aqueous cosmetic compositions comprise at least one polyol and at least one antioxidant.

In one aspect, the preservative-free aqueous cosmetic compositions comprise at least one acid and at least one ferment.

In one aspect, the preservative-free aqueous cosmetic compositions comprise distilled water and one or more components selected from propanediol, glycerin, medium-chain triglycerides, fatty alcohols, alkanes, polymers and copolymers, probiotic ferment (e.g., lactobacillus ferment), plant oils and wax, emollients, emulsifiers, citric acid, vitamins and antioxidants (e.g., sodium ascorbyl phosphate and tocopherol), skin-beneficial active ingredients such as sodium hyaluronate, ceramide, squalene, and essential fatty acids.

Other variations may be made without departing from the principles of the invention. For example, other ingredients can be used in place of distilled water, e.g., ultra-pure water, nano pure water, sterile water, bacteria filtered water, additional similar laboratory-grade water.

Other ingredients can also be used in place of the multifunctional ingredients of propanediol and glycerin, e.g., alcohols, various diols of pentylene glycol, propylene glycol, butylene glycol, etc., in the place of propanediol; other triols in the place of glycerin.

Other ferments can be used in the place of lactobacillus ferment, e.g., lactobacillus/soybean ferment extract, leuconostoc/radish root ferment filtrate, saccharomyces/ferment, Thermus thermophilus ferment, bifida ferment lysate, pseudoalteromonas ferment extract, saccharomyces/any metal (e.g., copper, zinc, magnesium, silicon, xylinum, etc.), alteromonas ferment extract and similar ferment and ferment lysates in the place of lactobacillus ferment.

Other acids can be used in the place of citric acid; acetic acid (vinegar), formic acid, trichloracetic acid, other similar acids. Cosmetic antioxidants are ingredients used in cosmetic formulations to increase the shelf life of cosmetic products and protect human cells against damage.

Other vitamins and antioxidants such as retinol (vitamin A), resveratrol, coenzyme Q10, polyphenols can be used in the place of niacinamide, vitamin C, and vitamin E. Many cosmetic ingredients known for skin benefits and numerous other fatty alcohols, esters and alkanes, polymers, and cosmetic emulsifiers can be added to the formulas.

Optional ingredients could also be provided in formulations of the invention, e.g., chelators, pH adjusters, and multifunctional ingredients with a secondary function of antimicrobial action. For example, any of numerous ingredients that bind with metal ions or metallic compounds, preventing them from adhering to a surface (such as skin) or causing contamination. Therefore, chelators such as tetrasodium EDTA and tetrahydroxypropyl ethylenediamine can be used additionally to enhance formulation emulsion stability and microstability. All of the individual components do not contain preservatives if they are in a blend or mixture form.

The preservative-free formulations described herein are “fresh” because they are not preserved and are designed to be used within a week to months from the manufacturing time point. This is different from cosmetic industry norms. The entire process of formulation manufacturing and packaging is conducted under cosmetic GMP (Good Manufacturing Practice). The formulation sample from the finished products has been tested for aerobic plate count (aka. total plate count). The aerobic plate count (APC) indicates the level of microorganisms and is a method of estimating the total number of microorganisms (mold, yeast, bacteria) in a product. Our finished products during the course of the supply chain and consumer use period were tested and passed both AOAC (the Association of Official Analytical Chemists) and microbial limit test USP 61 and 62 (General Chapter 61 and 62 of the United States Pharmacopeia (USP) National Formulary).

In one aspect, “preservative-free” means that compositions do not contain preservative chemicals listed in European Union Cosmetic Regulation (EU Cosmetic Products Regulation Annex V-Allowed Preservatives, https://ec.europa.cu/growth/tools-databases/cosing/pdf/COSING_Annex % 20V_v2.pdf.). This list includes substances designated as preservatives that cosmetic products marketed in the European Union may contain. A preservative is a natural or synthetic ingredient added to products to prevent them from spoiling. Preservatives are present in cosmetics to reduce the risk of microbial contamination of the product and ensure the product remains suitable and safe during shelf-life and the period of its use by consumers. The European Commission ensures an updated list of preservatives for their use in cosmetic products.

In another aspect, “preservative-free” means that formulations fail the antimicrobial effectiveness test (AET, aka, PET (preservative efficiency/challenge testing)) such as USP 51 or similar standard AET tests. USP 51 (General Chapter 51 of the United States Pharmacopeia (USP) National Formulary) is an antimicrobial test method that determines the antimicrobial efficacy of a material's antimicrobial preservatives. USP 51 is commonly used to evaluate the performance of preservatives used in cosmetics and personal care products.

In one aspect, the disclosed compositions meet the USP standard of free of microbes in the product for safe use. At the same time, the disclosed compositions avoid the use of typical preservatives both from synthetic and natural origin used in prior cosmetic compositions.

In one aspect, the disclosed compositions are in the form of a lotion, gel, or cream.

In one aspect, the disclosed compositions are suited for use with single dose-unit packaging.

In one aspect, the disclosed composition were found to improve skin conditions such as dry skin, itching, inflammatory skin lesions and other symptoms shown in the skin with eczema. These healing effects of the disclosed composition are due to being preservative-free because there is no or minimal irritation.

Exemplary preservative-free compositions according to the present disclosure are described below.

TABLE 1

| | Range | Alternative Range |
|---|---|---|
| Ingredient | % By Weight | % By Weight |
| Distilled Water | 20-90 | 50-70 |
| Diols and Triols | 2-15 | 5-10 |
| Fatty alcohols, esters, alkanes | 5-20 | 15-25 |
| Oils and Wax | 1-20 | 2-6 |
| Skin beneficial actives | 1-20 | 2.5-5 |
| Ferment | 1-50 | 2-4 |
| Vitamins and antioxidants | 0.5-5 | 1-2 |
| Acid | 0.1-10 | 0.15-0.5 |

### EXEMPLIFICATION

### Example 1

A cosmetic formulation was prepared in accordance with the present invention by first combining the Part A ingredients. The Part B ingredients were mixed in the main vessel and heating the mixture to 70-75° C. with agitation. The Part A mixture was added to Part B, and the temperature was maintained at 70-75° C., and mixing continued until a milky solution was obtained. The Part C ingredients were added to Part B while mixing and homogenized at 3000-4000 rpm for 3-5 minutes. The ABC mixture was mixed until uniform and was cooled down to 30-35° C. The Prat D was mixed and added to the ABC mixture at 30-35° C. The Part E ingredients were added slowly to the ABCD mixture, and further mixed and homogenized at 40° C. pH was adjusted using citric acid to 4.5-5.5. The batch was allowed to cool to room temperature.

The following table summarizes the ranges for the components of cosmetic formulations in accordance with the invention.

TABLE 2
Morning/AM Moisturizing Lotion

| | % by weight |
|---|---|
| | of total |
| Ingredient | composition |
| Part A | |
| Distilled Water | 10.0 |
| Biotin | 0.02 |
| Part B | |
| Distilled water | 50.0 |
| Glycerin | 2.5 |
| Propanediol | 5.0 |
| Ammonium Polyacryoyldimethyl Taurate | 0.5 |
| Panthenol | 0.5 |
| Part C | |
| Caprylic/Capric Triglyceride | 13.0 |
| Coco-Caprylate/Caprate | 4.0 |
| Cetearyl Alcocohol | 2.5 |
| Cetearyl Glucoside | 2.5 |
| Sodium Hyalrunonate | 0.5 |
| Glyceryl Stearate | 0.1 |
| Jojoba Esters | 0.2 |
| Sunflower Seed Wax | 1.0 |
| Sodium Stearoyl Lactylate | 0.1 |
| Rice Germ Extract | 1.0 |
| Cacao Seed Butter | 0.5 |
| Pycnogenol | 0.03 |
| Ceramide | 0.2 |
| Phytosphingosine | 0.2 |
| Glyceryl Caprylate | 0.1 |
| Part D | |
| Distilled Water | 2.5 |
| Niacinamide | 0.1 |
| Sodium Ascorbyl Phosphate | 0.1 |
| Green Tea Extract | 0.5 |
| Citric Acid | 0.15 |
| Part E | |
| Tocopherol | 0.2 |
| Lactobacillus Ferment | 2.0 |

### Example 2

A cosmetic formulation was prepared in accordance with the present invention by first combining the Part A ingredients. The Part A ingredients were mixed in the main vessel and heating the mixture to 70-75° C. with agitation. In a separate vessel, the Part B mixture ingredients were mixed at 70-75° C. until the mixture was uniform. The Part B mixture was added to the Part A, and the Part AB mixture was mixed until the mixture was uniform at 70-75° C. The AB mixture was then cooled down to 30-35° C. The Part C ingredients were mixed until dissolved and added to the Part AB for further mixing at 40-45° C. The Part D ingredients are added individually to the ABC mixture and was mixed thoroughly. The Part E was added to the Part ABCD mixture and further mixed and homogenized at 5000-6000 rpm until the ABCDE mixture was unform. pH was adjusted to 4.5-5.5. The batch was allowed to cool to room temperature.

The following table summarizes the ranges for the components of cosmetic formulations in accordance with the invention.

TABLE 3
Evening/PM Moisturizing Cream

| | % by weight of total |
|---|---|
| Ingredient | composition |
| Part A | |
| Distilled water | 63.08 |
| Glycerin | 2.5 |
| Propanediol | 5.0 |
| Part B | |
| Caprylic/Capric Triglyceride | 8.0 |
| Coco-Caprylate/Caprate | 2.5 |
| Cetearyl Alcocohol | 4.5 |
| Cetearyl Glucoside | 2.0 |
| Glyceryl Stearate | 1.0 |
| Jojoba Esters | 1.0 |
| Sunflower Seed Wax | 1.0 |
| Sodium Stearoyl Lactylate | 0.1 |
| Sweet Almond Oil | 0.5 |
| Olive Oil | 0.5 |
| Cacao Seed Butter | 0.5 |
| Ferulic Acid | 0.5 |
| Squalane | 2.5 |
| Linoleic Acid | 0.01 |
| Linolneic Acid | 0.01 |
| Phyoshingosine | 0.2 |
| Ceramide | 0.2 |
| Lycium Barbarum Callus Culture extract | 0.4 |
| Part C | |
| Distilled Water | 1.0 |
| Sodium Ascorbyl Phosphate | 0.1 |
| Part D | |
| Tocopherol | 0.2 |
| Lactobacillus Ferment | 2.0 |
| Part E | |
| Ammonium Polyacryoyldimethyl Taurate | 0.7 |

### Skin Healing

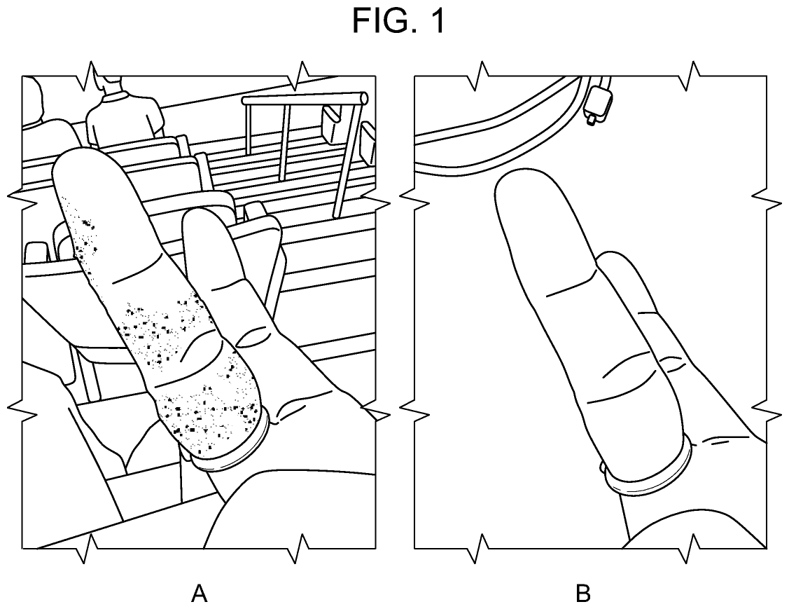

Eczema is a chronic skin disease characterized by dry skin, intense itching. inflammatory skin lesions, and a considerable impact on quality of life. Unfortunately, people with eczema (aka atopic dermatitis) often have difficulty using conventional moisturizers or market skincare products due to stinging sensations and irritation in response to certain ingredients contained in those products. Preservatives are one of the ingredients that people react to because preservatives are known to be skin irritants and sensitizers to create skin allergies. Hence, the symptoms can get aggravated by irritation from those conventional products. After using the disclosed compositions for 4 weeks, the subject showed skin healing and improved skin conditions. See FIG. 1A and FIG. 1B This subject did not observe similar healing benefits from other market moisturizers or skincare products.

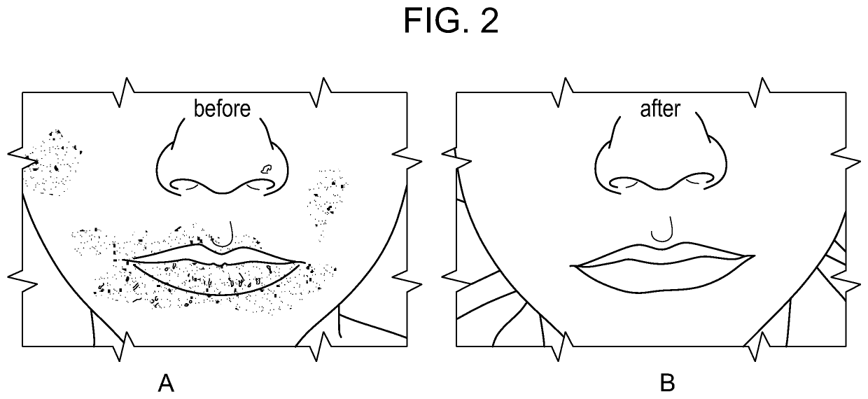

Similarly. after using the disclosed compositions for 2 weeks on a subject with extremely sensitive skin, the subject showed skin healing and improved skin conditions. See FIG. 2A and FIG. 2B.

### Formulation Emulsion Stability and Microstability

Emulsion stability studies were conducted on the disclosed compositions following the principles of the CTFA and COLIPA stability guidelines (March 2004). The compositions were packaged in 4 oz. glass jars and also in 0.8 mL sachets for single-dose units. They were examined for color, odor, appearance, viscosity and pH using the following test conditions:

TABLE 4
Temperature Conditions

| | Test Period | | | | | *F/T |
|---|---|---|---|---|---|---|
| | 3X F/T | 5° C. | 25° C. | 40° C. | 50° C. | 3X |
| Accelerated | 1 wk | X | X | X | X | |
| Stability | 2 wk | X | X | X | | |
| Program | 3 wk | X | X | X | | |
| | 4 wk | X | X | X | | |
| | 6 wk | X | X | X | | |
| | 8 wk | X | X | X | | |
| | 12 wk | X | X | X | | |

*F/T = Freeze and Thaw Cycles

The compositions were also stored under ambient room temperature conditions and tested one year after manufacturing. The compositions were consistent in structure and integrity. Changes in formulation color, odor, appearance, viscosity, and pH were minimal and considered not perceivable to consumers. Based on our experience during product development, this product will be stable for at least 30 months under normal storage conditions.

For microbial testing, the compositions were packaged in 0.8 mL sachets for single-dose units and then stored in accelerated conditions of 45° C. for three months. The temperature (45° C.) simulates the formulations' aging and promotes microbes' growth. They were tested at various time points (T=0, 1, 2, 4, 8 and 12 weeks) for the content of total aerobic microbial content (TAMC, bacteria) and total yeast and mold counts (TYMC) using a modified USP61 Total Plate Count Assay. Additionally, they were tested for pathogens using a modified USP62 Specified Microorganism Test. The compositions were also stored under ambient room temperature conditions and tested one year after manufacturing. The results show that the compositions are microbes-free, although the formulations were made without any preservatives. The formulations have a specification of <10 CFU/g or ml for all bacteria, yeast and mold and are absent of generally recognized pathogens (Escherichia coli, Staphylococcus aureus, and Pseudomonas aeruginosa) at all measured times.

1. A preservative-free aqueous cosmetic composition comprising distilled water.

2. The composition of claim 1, wherein the composition is in the form of an oil/water, water/oil, water-oil-water, or oil-water-oil emulsion.

3. The composition of claim 1 or 2, wherein the composition comprises at least one polyol.

4. The composition of any one of claims 1 to 3, wherein the composition comprises propanediol.

5. The composition of any one of claims 1 to 4, wherein the composition comprises at least one antioxidant.

6. The composition of any one of claims 1 to 5, wherein the composition comprises sodium ascorbyl phosphate or tocopherol.

7. The composition of any one of claims 1 to 6, wherein the composition comprises at least one acid.

8. The composition of any one of claims 1 to 7, wherein the composition comprises citric acid.

9. The composition of any one of claims 1 to 8, wherein the composition comprises at least one ferment.

10. The composition of any one of claims 1 to 9, wherein the composition comprises lactobacillus ferment.

11. The composition of any one of claims 1 to 10, wherein the distilled water is present at a concentration greater than or equal to 20% relative to the total weight of the composition.

12. The composition of any one of claims 1 to 11, wherein the composition is for single use.

13. The composition of any one of claims 1 to 12, wherein the composition fails preservative efficiency/challenge testing.

14. A method of treating a skin condition comprising applying the composition of any one of claims 1 to 13.

15. The method of claim 14, wherein the skin condition is selected from dry skin, itching, inflammatory skin lesions, and eczema.
